(19) United States
(12) Patent Application Publication
(10) Pub. No.: US 2023/0282126 A1
(43) Pub. Date: Sep. 7, 2023

(54) Methods and Systems for a Conflict Resolution Simulator

(71) Applicant: Smarter Reality, LLC, Round Rock, TX (US)

(72) Inventor: Walter Franklin Coppersmith, III, Round Rock, TX (US)

(73) Assignee: Smarter Reality, LLC, Round Rock, TX (US)

(21) Appl. No.: 18/050,456

(22) Filed: Oct. 27, 2022

Related U.S. Application Data
(60) Provisional application No. 63/315,828, filed on Mar. 2, 2022.

(51) Int. Cl. G09B 5/02; G06F 3/16; G10L 15/22; G06F 40/35; G10L 15/18; G06F 3/04847

(52) U.S. Cl. CPC G09B 5/02; G06F 3/167; G10L 15/22; G06F 40/35; G10L 15/1815; G06F 3/165; G06F 3/04847

(57) ABSTRACT

A method disclosed herein includes providing a user interface to a computing device, where the user interface presents a plurality of scenarios and each scenario of the plurality of scenarios is associated with dialogue with an AI virtual companion on a training topic. The method further includes receiving a selection of a scenario of the plurality of scenarios, receiving a verbal input associated with the scenario spoken by the user from the computing device, converting the verbal input to a textual representation, performing natural language processing on the textual representation to generate a natural language understanding result, determining a response to the verbal input, and controlling visual content associated with the scenario being rendered on the display of the computing device by rendering a representation of the AI virtual companion enacting the response.

### CROSS-REFERENCES TO RELATED APPLICATIONS

This application claims priority to and the benefit of U.S. Provisional Application Patent Ser. No. 63/315,828 filed Mar. 2, 2022, the entire disclosure of which is hereby incorporated by reference.

### TECHNICAL FIELD

This disclosure relates to simulation. More specifically, this disclosure relates to a system and method for a conflict resolution simulator.

### BACKGROUND

A number of cadets at military academies are accused of honor violations each year. Oftentimes, cadets lack communication skills to challenge peer behavior when witnessing honor violations. Further, many cadets and/or civilians lack the interpersonal skills to resolve conflicts in a civilized and/or efficient manner without letting their emotions get in the way.

### SUMMARY

Representative embodiments set forth herein disclose various techniques for enabling a conflict resolution simulator.

In one embodiment, a method for using dialogue simulations for training is disclosed. The method includes providing a user interface to display on a display of a computing device of a user, where the user interface presenting a plurality of scenarios and each scenario of the plurality of scenarios associated with dialogue with an artificially intelligent (AI) virtual companion pertaining to a training topic, receiving a selection of a scenario of the plurality of scenarios from the computing device, and receiving a verbal input associated with the scenario spoken by the user from the computing device. The method further includes converting the verbal input to a textual representation, performing natural language processing on the textual representation to generate a natural language understanding result, and determining, based on the natural language understanding result, a response to the verbal input, where the response including a dialogistic component and a behavioral characteristic of the AI virtual companion. Finally, the method includes controlling visual content associated with the scenario being rendered on the display of the computing device by rendering a representation of the AI virtual companion enacting the response.

In some embodiments, a tangible, non-transitory computer-readable medium stores instructions that, when executed, cause a processing device to perform any of the methods disclosed herein.

In some embodiments, a system includes a memory device storing instructions and a processing device communicatively coupled to the memory device. The processing device executes the instructions to perform any of the methods disclosed herein.

Other technical features may be readily apparent to one skilled in the art from the following figures, descriptions, and claims.

### BRIEF DESCRIPTION OF THE DRAWINGS

For a detailed description of example embodiments, reference will now be made to the accompanying drawings in which:

FIG. 1 illustrates a high-level component diagram of an illustrative system architecture according to certain embodiments of this disclosure;

FIG. 2 illustrates an example user interface for a starting screen of the conflict resolution simulator according to certain embodiments of this disclosure;

FIG. 3 illustrates an example user interface for a scenario selection screen according to certain embodiments of this disclosure;

FIG. 4 illustrates an example user interface for reviewal of user dialogue selections according to certain embodiments of this disclosure;

FIG. 5 illustrates an example user interface for comparing user choices and outcomes with other users according to certain embodiments of this disclosure;

FIG. 6 illustrates an example user interface for modifying and creating scenarios according to certain embodiments of this disclosure;

FIG. 7 illustrates example operations of a method for using dialogue simulations for training according to certain embodiments of this disclosure;

FIG. 8 illustrates a high-level component diagram of an illustrative system architecture according to certain embodiments of this disclosure;

FIG. 9 illustrates example operations of a method for using dialogue simulations for training with a computing device according to certain embodiments of this disclosure;

FIG. 10 illustrates an example user interface for a starting screen of the conflict resolution simulator according to certain embodiments of this disclosure;

FIG. 11 illustrates an example user interface for a scenario execution screen according to certain embodiments of this disclosure;

FIG. 12 illustrates another example user interface for a scenario execution screen according to certain embodiments of this disclosure;

FIG. 13 illustrates another example user interface for a scenario execution screen according to certain embodiments of this disclosure; and

FIG. 14 illustrates an example computer system.

### NOTATION AND NOMENCLATURE

Various terms are used to refer to particular system components. Different entities may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

The terminology used herein is for the purpose of describing particular example embodiments only, and is not intended to be limiting. As used herein, the singular forms “a,” “an,” and “the” may be intended to include the plural forms as well, unless the context clearly indicates otherwise. The method steps, processes, and operations described herein are not to be construed as necessarily requiring their performance in the particular order discussed or illustrated, unless specifically identified as an order of performance. It is also to be understood that additional or alternative steps may be employed.

The terms first, second, third, etc. may be used herein to describe various elements, components, regions, layers and/or sections; however, these elements, components, regions, layers and/or sections should not be limited by these terms. These terms may be only used to distinguish one element, component, region, layer or section from another region, layer or section. Terms such as “first,” “second,” and other numerical terms, when used herein, do not imply a sequence or order unless clearly indicated by the context. Thus, a first element, component, region, layer or section discussed below could be termed a second element, component, region, layer or section without departing from the teachings of the example embodiments. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C. In another example, the phrase “one or more” when used with a list of items means there may be one item or any suitable number of items exceeding one.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), solid state drives (SSDs), flash memory, or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

Definitions for other certain words and phrases are provided throughout this patent document. Those of ordinary skill in the art should understand that in many if not most instances, such definitions apply to prior as well as future uses of such defined words and phrases.

### DETAILED DESCRIPTION

Embodiments described herein are directed to a conflict resolution simulator (“simulator”) that is a virtual, interactive dialogue-driven training platform. The simulator may comprise an adaptive conversation engine, a high-fidelity AI virtual companion, a user-friendly conversation creation tool, a conversation library (where user-generated and supplied content can be accessed, shared, and customized), and a post-conversation analytics system. The simulator enables users to improve communication and collaboration by rehearsing simulated conversations on critical training topics with an artificially intelligent (AI) virtual companion. The simulator may provide an individualized, virtual experience by use of a customized video game engine that may power software with hundreds, thousands, millions, etc. of users concurrently via one or more sessions. The AI virtual companion may include customizable behaviors, goals, and mannerisms and play the role of cadet, co-worker, supervisor, or subordinate on various training topics. For example, the training topics may be related to honor (ethics), diversity and inclusion (D&I), and leadership (mentoring), among other things.

The following description focuses mainly on military scenarios. However, it should be noted that civilian scenarios are also included in the scope of this disclosure. For example, many professions, such as police officers, security guards, teachers, managers, and the like may benefit from the simulated conversation on topics related to honor (ethics), diversity and inclusion (D&I), and leadership (mentoring), etc. Any scenario may be adapted for relevancy to a particular profession.

At the US Air Force Academy (USAFA), more than two hundred cadets are accused of honor violations each year and often cadets lack communication skills to challenge peer behavior. Military academies or officer training schools may use the simulator to train and prepare cadets, officer trainees, and faculty to conduct difficult leadership conversations, such as potential honor violations. Using game and simulation technology, the simulator may train cadets on recognizing honor issues, appropriately confronting dishonorable behavior, and reducing the incidence of violations by encouraging early and effective individual engagement via pattern-matching from the simulator experience. Further, to reduce this number of honor violations, the simulator may be used to allow cadets to practice high-stress one on one leadership conversations (such as honor), create “just in time” training to address issues (such as the pandemic), provide confidential feedback to improve decision-making, and increase emotional intelligence via diversity and inclusion scenarios that expose cadets to diverse backgrounds and beliefs.

When using the simulator, users may login to the simulator using a computing device (e.g., a personal computer, mobile device, virtual reality device, augmented reality device etc.). The user may then select a conversation topic (e.g., cheating, stealing, lying, etc.) from a variety of learning objectives and receive an overview of the situation (e.g., by reviewing relevant documentation such as a plagiarized paper, reading investigatory reports, or watching a witness video) and desired outcomes. The user may begin conversing with one or more AI virtual companions by selecting from a variety of conversational topics or comments. The AI virtual companion may respond based on the user's attitude (e.g., accusatory, friendly, angry, etc.) and conversational choices. Further, the AI virtual companion's programmed characteristics and scenario background influences the AI virtual companion's behavior and the course of the virtual conversation. Once the scenario is complete, the user can review their choices and play the scenario again, making different choices for a different outcome.

The AI virtual companion provides realistic conversational behavior with customizable attributes (attitude, goals, and mannerism). The interactive conversation provides different participant choices and leads to different outcomes (e.g., users can replay and make different choices). Other features of the simulator may include: multiple, diversified virtual companions across cadet demographics; adjustable “behavioral” elements so that players face a range of emotional responses (e.g., angry, sullen, quiet); realistic facial expressions and non-verbal cues; conversations pulled directly from real-life cadet honor violations, locations and interpersonal challenges; and re-playable experiences by adjusting the virtual companion's base behavior, user choices, and scenario background, among other things.

Some benefits of the embodiments described herein include an AI system that provides realistic conversational behavior with customizable attributes (e.g., attitude, goals and mannerism) and interactive conversation with different participant choices leading to different outcomes (e.g., replay and make different choices). Another benefit includes a customizable platform including customization of facts of the scenarios, AI behavioral attributes, and starting conditions of the scenarios that can be adjusted to suit training needs. Another benefit of the embodiments include its iterative nature where participants can “play, fail fast, and learn” to experiment with different approaches and ideas without risk. Still yet, other benefits of the embodiments include: analytical tools that allow users to compare choices and outcomes with others in the community anonymously to understand shared values; a user-friendly scenario creator allowing “just in time” solutions from cadets and faculty; and sharable and customizable scenarios via an integrated “library” of user-created scenarios. Additionally, the embodiments described herein employ user interfaces that mirrors well-known video game experiences for ease of adoption, high fidelity visuals that enables the user to recognize non-verbal cues to the AI-companion's mental state, and well-understood game mechanics.

In some embodiments, the present disclosure provides various technical solutions to technical problems. The technical problems may include providing virtual simulations of scenarios based on user input (e.g., speech, gesture, vital signs, etc.), and real-time control of the AI virtual companion in response to the user input. The technical solution may include receiving the user input via one or more input peripherals (e.g., microphone, vibration sensor, pressure sensor, camera, etc.) and use speech-to-text conversion and natural language processing techniques to transform the speech to text and to use one or more machine learning models trained to input the text and output a meaning of the text. The meaning of the text may be used by an expert AI system to determine one or more reactions to meaning, and the one or more reactions may be used to control the AI virtual companion presented digitally in a display screen of a virtual reality device. Such techniques may provide technical benefits of dynamically adjusting reactions of an AI virtual companion within a virtual reality device in real-time based on transformed user input (e.g., audible spoken words transformed into text that is interpreted via natural language processing).

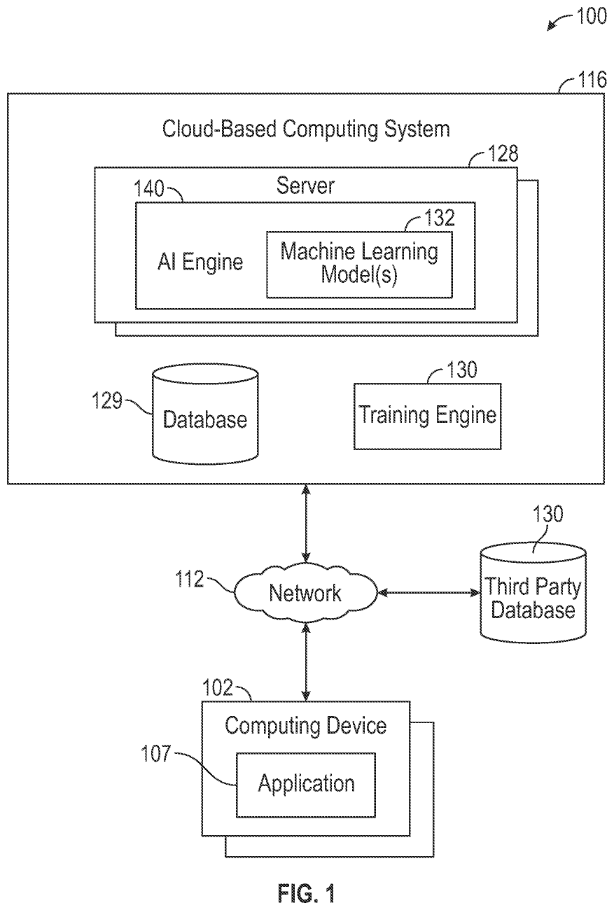

To explore the foregoing in more detail, FIG. 1 will now be described. FIG. 1 illustrates a high-level component diagram of an illustrative system architecture 100 according to certain embodiments of this disclosure. In some embodiments, the system architecture 100 may include computing devices 102, a cloud-based computing system 116, and/or a third party database 130 that are communicatively coupled via a network 112. As used herein, a cloud-based computing system refers, without limitation, to any remote or distal computing system accessed over a network link. Each of the computing devices 102 may include one or more processing devices, memory devices, and network interface devices.

The network interface devices of the computing devices 102 may enable communication via a wireless protocol for transmitting data over short distances, such as Bluetooth, ZigBee, near field communication (NFC), etc. Additionally, the network interface devices may enable communicating data over long distances, and in one example, the computing devices 102 may communicate with the network 112. Network 112 may be a public network (e.g., connected to the Internet via wired (Ethernet) or wireless (WiFi)), a private network (e.g., a local area network (LAN), wide area network (WAN), virtual private network (VPN)), or a combination thereof.

The computing device 102 may be any suitable computing device, such as a laptop, tablet, smartphone, virtual reality device, augmented reality device, or computer. The computing device 102 may include a display that is capable of presenting a user interface of an application 107. As one example, the computing device 102 may be operated by cadets or faculty of a military academy. The application 107 may be implemented in computer instructions stored on a memory of the computing device 102 and executed by a processing device of the computing device 102. The application 107 may be a conflict resolution platform including an AI-enabled simulator and may be a stand-alone application that is installed on the computing device 102 or may be an application (e.g., website) that executes via a web browser. The application 107 may present various screens, notifications, and/or messages to a user. The screens, notifications, and/or messages may be associated with dialogue with an AI virtual companion on a training topic.

In some embodiments, the cloud-based computing system 116 may include one or more servers 128 that form a distributed, grid, and/or peer-to-peer (P2P) computing architecture. Each of the servers 128 may include one or more processing devices, memory devices, data storage, and/or network interface devices. The servers 128 may execute an AI engine 140 that uses one or more machine learning models 132 to perform at least one of the embodiments disclosed herein. The servers 128 may be in communication with one another via any suitable communication protocol. The servers 128 may enable configuring a scenario for a user on a training topic. For example, the training topics may be related to one or more of the following topics: honor, diversity and inclusion, and leadership. The servers 128 may provide user interfaces that are specific to a scenario. For example, a user interface provided to the user may include background information on the scenario. The servers 128 may execute the scenarios and may determine inputs and options available for subsequent turns based on selections made by users in previous turns. The servers 128 may provide messages to the computing devices of the users participating in the scenario. The servers 128 may provide messages to the computing devices of the users after the scenario is complete. Additionally, AI engine 140 may include the conflict resolution simulator. The conflict resolution simulator comprise the following components: an adaptive conversation engine, a high-fidelity AI virtual companion, a user-friendly conversation creation tool, a conversation library (where user-generated and supplied content can be accessed, shared, and customized), and a post-conversation analytics system.

In some embodiments, the cloud-based computing system 116 may include a database 129. The cloud-based computing system 116 may also be connected to a third party database 130. The databases 129 and/or 130 may store data pertaining to scenarios, users, results of the scenarios, and the like. The results may be stored for each user and may be tracked over time to determine whether a user is improving. Further, observations may include indications of which types of selections are successful in improving the success rate of a particular scenario. Completed scenarios including user selections taken and responses to the user selections for each turn in the scenarios may be saved for subsequent playback. For example, a user may review the saved completed scenario to determine what were the right and wrong user selections taken by the user during the scenario. The database 129 or 130 may store a library of scenarios that enable the users to select the scenarios and/or share the scenarios.

The computing system 116 may include a training engine 130 capable of generating one or more machine learning models 132. Although depicted separately from the AI engine 140, the training engine 130 may, in some embodiments, be included in the AI engine 140 executing on the server 128. In some embodiments, the AI engine 140 may use the training engine 130 to generate the machine learning models 132 trained to perform inferencing operations, predicting operations, determining operations, controlling operations, or the like. The machine learning models 132 may be trained to simulate a scenario based on user selections and responses, to dynamically update user interfaces for scenarios and specific turns based on one or more user selections (e.g., dialogue options) in previous turns, to dynamically update user interfaces by changing available information (e.g., dialogue), to select the responses, available information, and next state of the scenario in subsequent turns based on user selections and combination of user selections in previous turns, and/or to improve feature selection of the machine learning models 132 by scoring the results of the scenarios produced, among other things. The one or more machine learning models 132 may be generated by the training engine 130 and may be implemented in computer instructions executable by one or more processing devices of the training engine 130 or the servers 128. To generate the one or more machine learning models 132, the training engine 130 may train the one or more machine learning models 132.

The training engine 130 may be a rackmount server, a router, a personal computer, a portable digital assistant, a smartphone, a laptop computer, a tablet computer, a netbook, a desktop computer, an Internet of Things (IoT) device, any other desired computing device, or any combination of the above. The training engine 130 may be cloud-based, be a real-time software platform, include privacy software or protocols, or include security software or protocols.

To generate the one or more machine learning models 132, the training engine 130 may train the one or more machine learning models 132. The training engine 130 may use a base data set of user selections and scenario states and outputs pertaining to resulting states of the scenario based on the user selections. In some embodiments, the base data set may refer to training data and the training data may include labels and rules that specify certain outputs occur when certain inputs are received. For example, if user selections are made in turn 2, then certain responses/states of the scenario and user interfaces are to be provided in turn 3.

The one or more machine learning models 132 may refer to model artifacts created by the training engine 130 using training data that includes training inputs and corresponding target outputs. The training engine 130 may find patterns in the training data wherein such patterns map the training input to the target output and generate the machine learning models 132 that capture these patterns. Although depicted separately from the server 128, in some embodiments, the training engine 130 may reside on server 128. Further, in some embodiments, the artificial intelligence engine 140, the database 150, or the training engine 130 may reside on the computing device 102.

As described in more detail below, the one or more machine learning models 132 may comprise, e.g., a single level of linear or non-linear operations (e.g., a support vector machine (SVM) or the machine learning models 132 may be a deep network, i.e., a machine learning model comprising multiple levels of non-linear operations. Examples of deep networks are neural networks, including generative adversarial networks, convolutional neural networks, recurrent neural networks with one or more hidden layers, and fully connected neural networks (e.g., each artificial neuron may transmit its output signal to the input of the remaining neurons, as well as to itself). For example, the machine learning model may include numerous layers or hidden layers that perform calculations (e.g., dot products) using various neurons. In some embodiments, one or more of the machine learning models 132 may be trained to use causal inference and counterfactuals.

For example, the machine learning model 132 trained to use causal inference may accept one or more inputs, such as (i) assumptions, (ii) queries, and (iii) data. The machine learning model 132 may be trained to output one or more outputs, such as (i) a decision as to whether a query may be answered, (ii) an objective function (also referred to as an estimand) that provides an answer to the query for any received data, and (iii) an estimated answer to the query and an estimated uncertainty of the answer, where the estimated answer is based on the data and the objective function, and the estimated uncertainty reflects the quality of data (i.e., a measure which takes into account the degree or salience of incorrect data or missing data). The assumptions may also be referred to as constraints and may be simplified into statements used in the machine learning model 132. The queries may refer to scientific questions for which the answers are desired.

The answers estimated using causal inference by the machine learning model may include optimized scenarios that enable more efficient training of military personnel. As the machine learning model estimates answers (e.g., scenario outcomes based on alternative action selection), certain causal diagrams may be generated, as well as logical statements, and patterns may be detected. For example, one pattern may indicate that “there is no path connecting ingredient D and activity P,” which may translate to a statistical statement “D and P are independent.” If alternative calculations using counterfactuals contradict or do not support that statistical statement, then the machine learning model 132 may be updated. For example, another machine learning model 132 may be used to compute a degree of fitness which represents a degree to which the data is compatible with the assumptions used by the machine learning model that uses causal inference. There are certain techniques that may be employed by the other machine learning model 132 to reduce the uncertainty and increase the degree of compatibility. The techniques may include those for maximum likelihood, propensity scores, confidence indicators, or significance tests, among others.

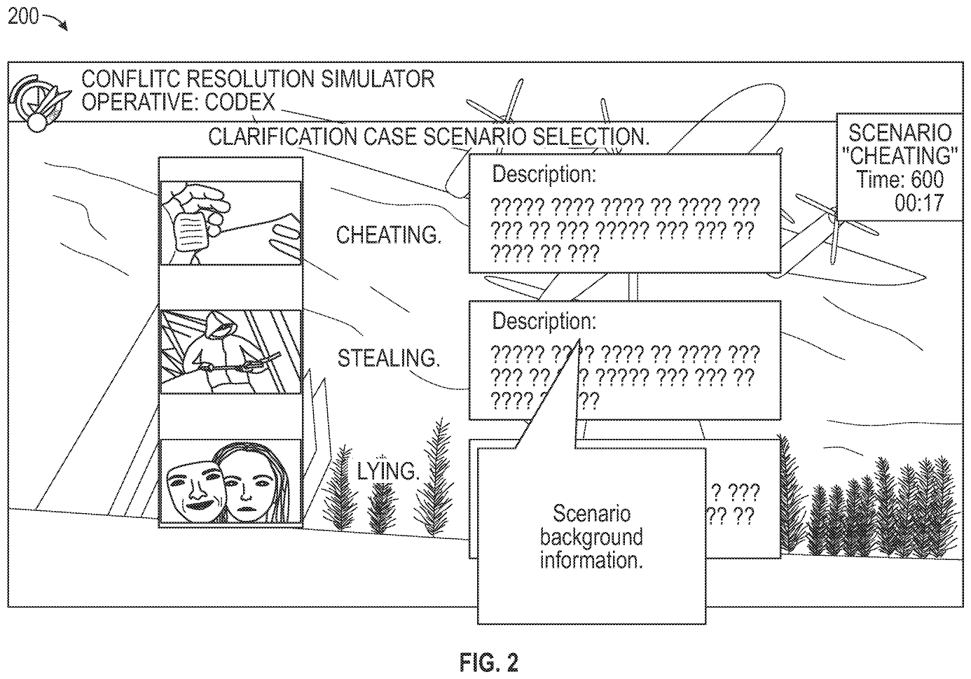

FIG. 2 illustrates an example user interface 200 for a starting screen of the conflict resolution simulator according to certain embodiments of this disclosure. The user interface 200 presents a scenario selection screen. The user interface 200 also includes various graphical elements (e.g., buttons) for different scenarios (e.g., cheating, stealing, lying, etc.). For example, the user may select from among multiple scenario options depending on their learning objective. As shown in FIG. 2, the user interface 200 may also display background information on the scenario. The background information may include a description of the scenario. The user interface 200 may be presented when a user logs into the conflict resolution simulator with his or her credentials. The selection of the scenario may be transmitted to the cloud-based computing system 116.

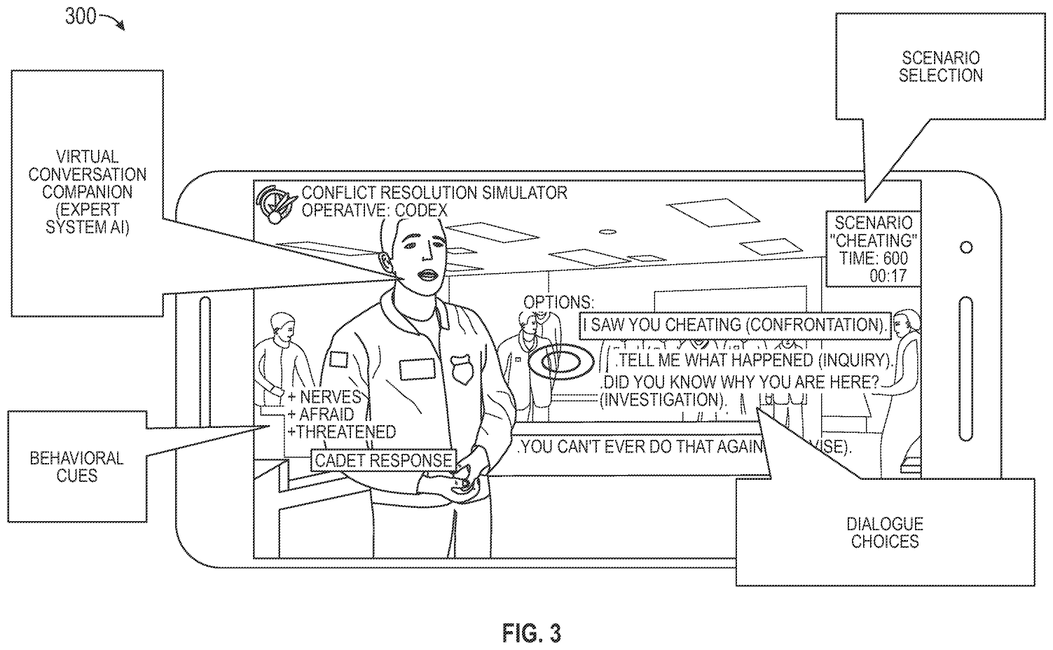

FIG. 3 illustrates an example user interface 300 for a scenario selection screen according to certain embodiments of this disclosure. As depicted, the user interface 300 presents an AI virtual companion. For example, after a cadet selects a scenario (e.g., cheating) in the user interface 200, the simulator starts. In some embodiments, the cadet may be initially given background information including “evidence” to review such as a homework assignment. For example, the user may be prompted to review relevant documentation such as a plagiarized paper, reading investigatory reports, or watching a witness video.

As further depicted in FIG. 3, the user may begin conversing with one or more virtual companions by selecting from a variety of conversational topics or comments (e.g., dialogue choices or options). For example, as shown in FIG. 3, the dialogue choice selected by the user may include “I saw you cheating (confrontation).” As depicted in FIG. 3, in one example scenario, the AI virtual companion is a cadet at a military academy who has been caught cheating and a user plays the role of a faculty member of the military academy. In another example scenario, the AI virtual companion is a cadet at a military academy who has been caught cheating and the user plays the role of a peer and fellow cadet at the military academy.

In some embodiments, the AI virtual companion may respond based on the user's attitude (e.g., accusatory, friendly, angry, etc.) and conversational choices. Further, the AI virtual companion's programmed characteristics and scenario background influences the AI virtual companion's behavior and the course of the virtual conversation. As further shown in FIG. 3, the AI virtual companion may also display different behavioral cues (e.g., nerves, afraid, threatened, etc.). In some embodiments, the AI virtual companion may include adjustable “behavioral” elements so that users face a range of emotional responses (e.g., angry, sullen, quiet). The cloud-based computing system 116 may receive the user selections and the AI engine 140 may begin the simulation of the scenario with customized user interfaces for each user selection and each turn, where the user interfaces are dynamically modified in subsequent turns based on the user selections in previous turns.

Additionally, FIG. 3 illustrates user interface 300 of the simulator executing on a mobile device according to certain embodiments of this disclosure. As depicted, various graphical elements may be used to display information and simultaneously prompt a user to select different dialogue options during a turn of the scenario presented on the user interface 300. The various graphical elements may enable presenting relevant information in a manner that does not inundate the small screen of the mobile device. Accordingly, the user interface 300 provides an enhanced experience for users using the simulator.

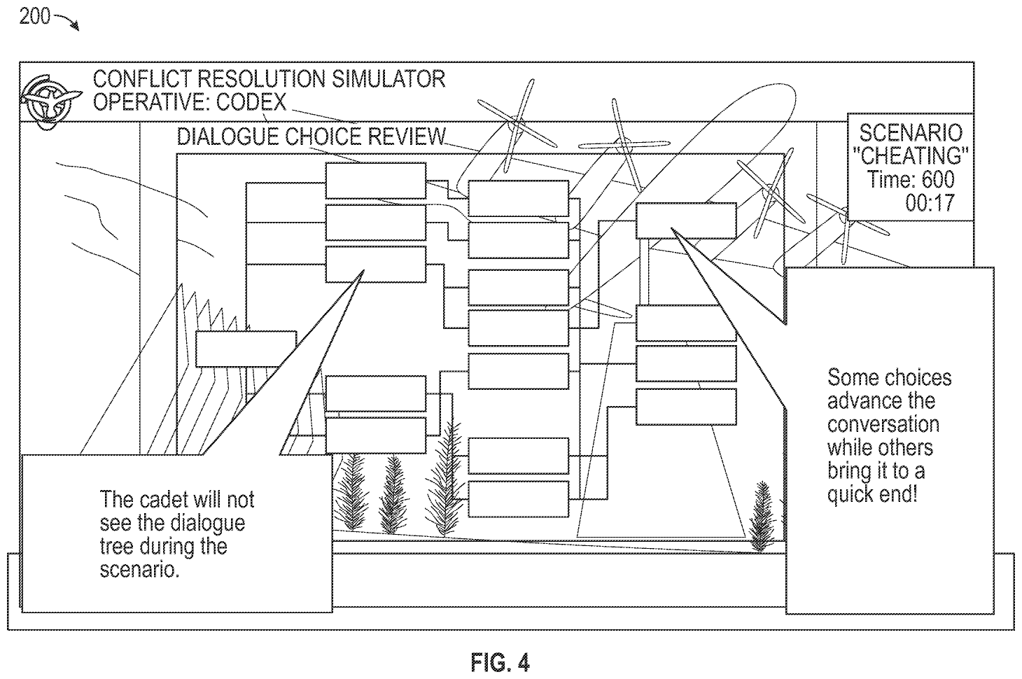

FIG. 4 illustrates an example user interface 400 for reviewing a user's dialogue selections according to certain embodiments of this disclosure. For example, once the scenario is complete, the user can review their choices and play the scenario again, making different choices for a different outcome. In some embodiments, as the cadet makes different dialogue selections, the simulator moves through a set of decision-trees and presents the cadet with new information related to the training topic. Some user selections may advance the conversation, while other user selections may end the conversation. In some embodiments, using user interface 400, the user may playback the scenario and review selections made by the user during the scenario.

For example, in a scenario related to cheating, where the AI virtual companion is a cadet at a military academy who has been caught cheating and a user plays the role of a faculty member of the military academy, example dialogue options (i.e., faculty responses) for the scenario for the faculty member are provided in the table below. Additionally, as depicted below, dialogue options are dependent based on attributes (e.g., a cadet being adversarial) of the AI virtual companion. As indicated, some dialogue options are categorized as “good” and others are categorized as “slightly off center.”

| Common excuse used by | | Faculty Responses - Slightly |
|---|---|---|
| cadets: | Faculty Responses - Good | Off Center |
| Perhaps the MOSS software | Since cadets are all working | Since cadets are all working on |
| just picked up the similarities | on the same assignment, it is | the same assignment, it is |
| in our codes because there are | normal to have a small | normal to have a small number |
| only a few ways to complete | number of similarities to the | of similarities to the rest of |
| the assignment. Cadets will | rest of cadets in the class. | cadets in the class. But that is |
| naturally have similar | However, it is very unlikely | not what we have here. The high |
| responses if they follow your | that such similar code | degree of similarity shows me |
| assignment directions. I don't | between two cadets would | this was not random and that |
| know how else this could | occur by chance. I would like | you used another cadet's |
| happen. | to understand how this may | computer code to cheat. The |
| | have happened. Can you | Honor system favors cadets who |
| | explain your process for | are upfront and admit. This is |
| | completing this assignment | your chance to get on the right |
| | and include anyone you may | side. I'm trying to help you by |
| | have worked with or received | giving you a chance here to |
| | help from? Did you help any | admit and turn yourself in. |
| | other cadets complete their | |
| | assignment? | |

The cadet that tries to negotiate an academic penalty in exchange for not reporting to
honor:

| | | |
|---|---|---|
| I really had no idea my code | I cannot allow you to redo the | That sounds reasonable, but I |
| was so similar and don't | assignment. I would like to | would prefer that you |
| know how it happened. Can | understand how your code is | demonstrate that skill right now |
| you give me another chance | so similar though. Can you | in front of me. If you can do |
| to do the assignment, so I can | explain your process for | that, then I won't refer it to the |
| prove to you I know how to | completing this assignment | honor process but I'll still do an |
| do it and I didn't cheat? | and include anyone you may | academic penalty. |
| | have worked with or received | |
| | help from? Is there anyone | |
| | you helped with their | |
| | assignment? | |
| I am very confused and scared | The academic penalty process | Can you tell me why you think |
| because I never intended for | is completely separate from | you deserve an academic |
| this. I don't want to be put on | the honor process and does not | penalty and not refer this to the |
| honor for something I was | require intent. I am very | Honor system? If you are |
| completely unaware of. The | interested in talking about | admitting you deserve an |
| honor process is notorious for | intent here. It is unlikely that | academic penalty, why would |
| not accepting the fact that | two students' codes would be | you think that is also not an |
| there was truly no intent. | so similar by random chance. | Honor Code violation? |
| Could you potentially give me | I am concerned that you | |
| a zero, and I could redo the | worked with another cadet or | |
| assignment to prove to you | shared code. For example, you | |
| that I can do it? | both used the same variable | |
| | names throughout the | |
| | assignment. No other student's | |
| | chose variable names that | |
| | match someone else. Do you | |
| | know why your variable | |
| | names are the same as another | |
| | cadet's? | |
| Please just give me a zero on | I understand, this is very | I understand, this is very |
| the assignment and leave it out | stressful. And having a | stressful. And having a previous |
| of honor. I already have one | previous honor violation does | honor violation does make it |
| honor hit and they will kick | make it more stressful. | more stressful. Despite bringing |
| me out if I get another. I | Despite bringing this | this suspicion to you, I am still |
| promise I've learned my | suspicion to you, I am still | your instructor and I value you |
| lesson already. | your instructor and I value you | and support you. As your |
| | and support you. I want to | instructor I have a responsibility |
| | address your specific | to ensure you learn the course |
| | concerns. I cannot substitute | material and that you develop as |
| | an academic penalty for | a future officer. I would like you |
| | referring to the honor process | to redo this assignment and in |
| | if I believe there is act and | addition, write me a one page |
| | intent. They are two separate | paper what you have learned |
| | processes. I would like to talk | from this situation. There is no |
| | more about the underlying act | need to draw this out further if |
| | and intent that brought us | you understand the course |
| | here. I am concerned for | objectives and learned from |
| | example, because you both | your mistakes. |
| | used the same variable names | |
| | throughout the assignment. | |
| | No other student's chose | |
| | variable names that match | |
| | someone else. Do you know | |
| | why your variable names are | |
| | the same as another cadet's? | |

Neither admit nor deny-cadet appeals to empathy for “stressful situation” and hopes the
case gets dropped, but hedges in case they want to admit later in the process:

| | | |
|---|---|---|
| I kind of see the similarities | The MOSS Software detected | What it means to me is that you |
| you are pointing out, but I | a high degree of similarity | used another cadet's computer |
| don't understand what it | between your code and one | code to cheat. The Honor |
| means. | other cadet's. It is unlikely that | system favors cadets who are |
| | such similar code between | upfront and admit. This is your |
| | two cadets would occur by | chance to get on the right side. |
| | random chance. I would like | I'm trying to help you by giving |
| | to understand how this may | you a chance to admit and turn |
| | have happened. Can you | yourself in. |
| | explain your process for | |
| | completing this assignment | |
| | and include anyone you may | |
| | have worked with, received | |
| | help from, and anyone you | |
| | helped complete their | |
| | assignment? | |
| I was up very late working on | I understand. Let's talk about | I'm so sorry to hear this. I've had |
| the assignment and I was sleep | what you're able to remember | a lot of late nights too, trust me. |
| deprived. It was a stressful | so I can make sure my | What's all the stuff going on in |
| time with lots of stuff in my | concerns are tackled head on. | your life? |
| life. I really don't remember | | |
| much, but cheating is | | |
| inconsistent with my | | |
| character. | | |
| A lot of problems are going on | Can you recall the process you | I agree it is unlikely a cadet on |
| at home this semester. I've | used to decide on variable | the Dean's list cheated. My |
| had trouble sleeping and | names in the assignment? I | guess is the other cadet cheated |
| focusing. I hope you can trust | am concerned for example, | off of you, maybe without your |
| me that I just can't remember | because you both used the | knowledge. Do you have any |
| much about doing the | same variable names | idea how someone else may |
| assignment. I've been on the | throughout the assignment. | have gotten access to your work |
| Dean's list every semester | No other student's chose | or ideas? |
| here. I'm not the type that | variable names that match | |
| cheats because I don't need to. | someone else. Do you know | |
| | why your variable names are | |
| | the same as another cadet's? | |

The rare, but occassional adversarial cadet:

| | | |
|---|---|---|
| This is supposed to be neutral | I am sorry you feel that way. I | I am sorry you feel that way, but |
| and impartial. You are | am trying, but not | I have a duty to uphold the |
| clearly not listening to my | understanding how your | Honor Code. I understand what |
| explanation. You seem to | explanation explains the | you are saying, but it doesn't |
| have your mind made up that | similarities in the code that I | explain or make sense of the |
| I cheated and nothing I am | am seeing. I think it would be | similarities between the |
| going to say is going to | best to end the clarification | computer codes. You are saying |
| change that. | and reconvene in a day or two. | you didn't cheat, but it is clear |
| | I will ask your Honor Officer | not all the computer code is |
| | to set up a formal clarification. | yours. Things would be a lot |
| | That will give each of us time | better if you just tell me the |
| | to organize our thoughts and | truth. The Honor system favors |
| | have a neutral Honor Officer | cadets who are upfront and |
| | present to help us sort this out. | admit. I'm trying to help you by |
| | | giving you a chance to admit |
| | | and turn yourself in. |

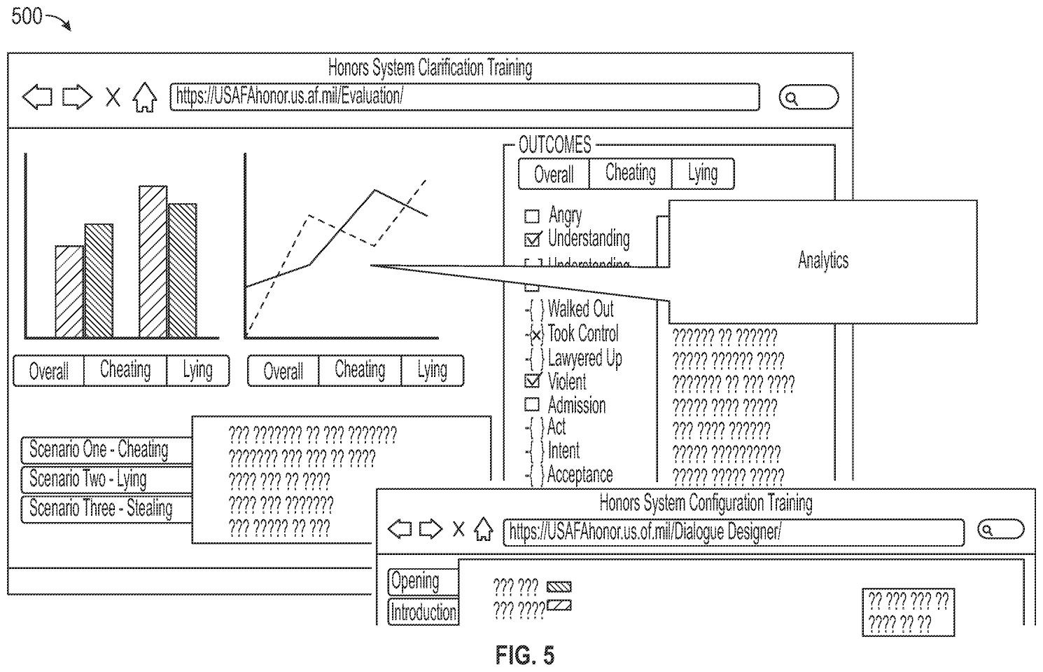

FIG. 5 illustrates an example user interface 500 for comparing choices and outcomes with other users according to certain embodiments of this disclosure. For example, users can compare choices and outcomes with other users anonymously to understand shared values within a community. As depicted, user interface 500 may display graphical representations of the performance of a scenario of the user and other users. In some embodiments, each user selection may be scored. For example, the table below provides an example embodiment of a dialogue options of a scenario and how the user selections of dialogue options may be scored. The field, “Entry,” represents a conversational choice for the user, the field, “Option,” represents choices the user can select, the field, “Text String,” represents what is displayed on the user interface for the user to select, the field, “Response Factor,” is the summation of the response factor that influences the behavior of the AI virtual companion, and the field, “Evaluation Factor” is related to representing each choice as a better or worse option. The evaluation factor also provide a way to show these options during post-conversation evaluation.

| | | | Response | Evaluation |
|---|---|---|---|---|
| Step | Option | Text String | Factor | Factor |
| 1 | a | Grab a seat. | −1 | 6 |
| | b | I have some concerns about your combined loading | −2 | 4 |
| | | lab assignment. | | |
| | c | Thanks for meeting with me. | 0 | 10 |
| | d | Did cadet X share code with you? | −3 | 2 |
| 2 | a | Today, I've asked to meet so we can talk about the | −1 | 10 |
| | | combined loading lab assignment. | | |
| | b | Relax, everything is going to be fine. | 0 | 6 |
| | c | Did you copy code from anyone? | −3 | 2 |
| | d | Do you know why you are here? | −2 | 4 |
| 3 | a | Did you read my email and understand what I was | −1 | 6 |
| | | asking? | | |
| | b | I think you may have copied some of the code from | −3 | 2 |
| | | another cadet. | | |
| | c | Do you know what “MOSS” is? | −2 | 4 |
| | d | I've got some questions and concerns about your turn | −1 | 10 |
| | | in. | | |
| 4 | a | I'm going to address my concerns with you and we'll | −1 | 8 |
| | | discuss them. | | |
| | b | This is an informal honor clarification in which I'll | −2 | 10 |
| | | address my concerns with you. | | |
| | c | I'm going to address my concerns with you and I need | −3 | 4 |
| | | you to answer truthfully and completely. | | |
| | d | Just answer my questions please. | −5 | 2 |
| 5 | a | In this course, we run all code through MOSS - | −1 | 10 |
| | | measure of software similarity tool. | | |
| | b | Your software is similar to another cadet's software | −2 | 8 |
| | | turn in. | | |
| | c | Why does your software look like cadet X's code? | −3 | 4 |
| | d | I think you copied cadet X's code. Did you? | −5 | 2 |
| 6 | a | MOSS shows me that you are cheating | −5 | 2 |
| | b | MOSS shows that your code looks like another | −1 | 8 |
| | | cadet's code. Why is that? | | |
| | c | MOSS shows that the code you submitted came from | −5 | 4 |
| | | somewhere else. Did you copy it? | | |
| | d | MOSS shows if code looks similar across different | −1 | 10 |
| | | cadets and it reported that yours does. | | |
| 7 | a | I think you used unauthorized resources in writing | −3 | 4 |
| | | your code. | | |
| | b | Authorized resources are the text, the professor and | −1 | 10 |
| | | MATLAB. Do you understand that? | | |
| | c | What resources did you use in writing your code? | 0 | 8 |
| | d | Did you see cadet X's code before turn in? | −3 | 2 |
| 8 | a | Here's a copy of your code turn in. Can you take a | 0 | 10 |
| | | look at this with me? | | |
| | b | Did you bring a copy of your code turn in? | 0 | 4 |
| | c | Here's a copy of your code turn in. Can you explain | 0 | 8 |
| | | it to me? | | |
| | d | Here's a copy of your code turn in. What parts did | −3 | 2 |
| | | you copy from cadet X? | | |
| 9 | a | You had extremely high similarity on MOSS with | −2 | 10 |
| | | another cadet's assignment | | |
| | b | My concern is that there is a high similarity of code | −2 | 10 |
| | | to this other cadet's assignment. | | |
| | c | Can you explain why there is similarity with another | −2 | 8 |
| | | cadet's code? | | |
| | d | I've talked to other cadets and I know you saw their | −3 | 6 |
| | | code before your turn in. | | |
| 10 | a | Can you explain why there is such a high similarity | −2 | 8 |
| | | between your code and another cadets? | | |
| | b | Can you walk me through your code and explain why | −2 | 10 |
| | | there is similarity? | | |
| | c | You copied this part of the code, didn't you? | −4 | 4 |
| | d | [say nothing] | 0 | 2 |
| 11 | a | Would other cadets tell me that they shared code with | −2 | 6 |
| | | you? | | |
| | b | Cadets X & Y told me they shared code with you. | −3 | 8 |
| | c | Cadet X said she sent you the code after the | −4 | 10 |
| | | assignment was completed. | | |
| | d | Did you turn your code in late because you wanted to | −5 | 4 |
| | | look at another's code? | | |
| 12 | a | I had a clarification with Cadet X and she admitted | −3 | 10 |
| | | she sent you her code. | | |
| | b | I plan to have a clarification with Cadet X. What will | −1 | 6 |
| | | she tell me? | | |
| | c | I plan to speak to many cadets about your behavior. | −2 | 4 |
| | d | Are you lying to me? | −4 | 2 |
| 13 | a | I'm just telling you what I know - you don't have to | −3 | 4 |
| | | be upset. | | |
| | b | I'm not sure you are telling me the truth. | −4 | 6 |
| | c | I'm being forthright with you, I hope you are being | −3 | 10 |
| | | forthright with me. | | |
| | d | I'm concerned that you are compounding your | −5 | 2 |
| | | cheating with lying to me. | | |
| 14 | a | I suspect a violation occurred. | −5 | 6 |
| | b | I see a strong similarity between the code and suspect | −5 | 10 |
| | | a violation occurred. | | |
| | c | I see a strong similarity between the code. | −5 | 8 |
| | d | I suspect that you copied the code in violation of the | −5 | 4 |
| | | honor code. | | |
| 15 | a | Do you have anything else to say? | −3 | 8 |
| | b | I'm going to talk to some other cadets. | −3 | 6 |
| | c | I am not satisfied with your explanation of why there | −5 | 10 |
| | | is such similarity of the code. | | |
| | d | We are going to proceed with a formal honor | −5 | 10 |
| | | clarification. | | |

The user interfaces described herein may be presented in real-time or near real-time. The selections made by the user using graphical elements of the user interfaces may be used by the AI engine 140 to determine the state of the scenario in the next turn and to generate the user interfaces that are presented for each turn of in the scenario. It should also be noted that different users may concurrently participate in different scenarios at the same time using the simulator.

By providing graphical representations of a user's performance in a scenario, the user can quickly evaluate his or her performance and determine if he or she needs additional training in a topic. Providing graphical representations for the scenario enables the user to make a decision quickly without having to drill-down and view each turn of the scenario in detail. Accordingly, the user interface 500 provides an enhanced experience for users using the simulator.

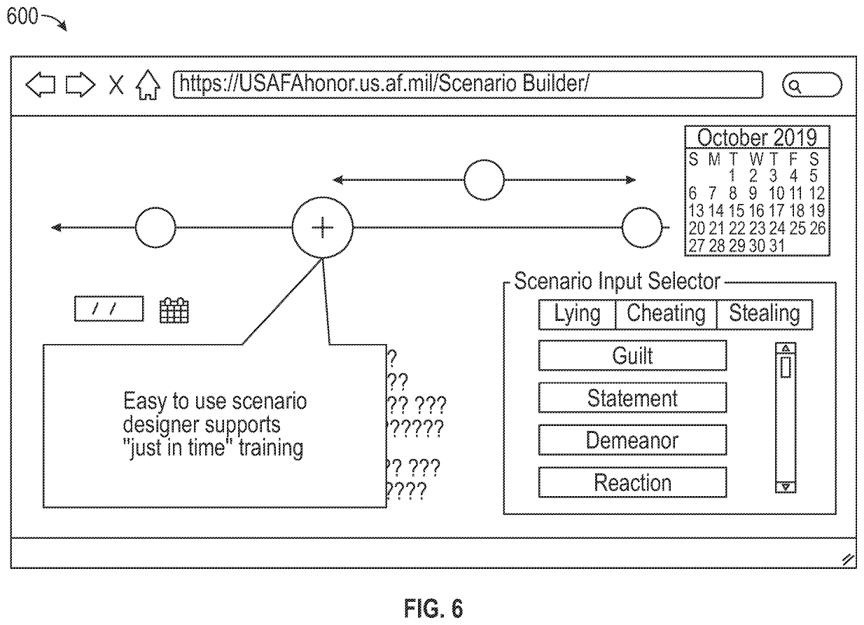

FIG. 6 illustrates an example user interface 600 for enabling a user to modify or create scenarios according to certain embodiments of this disclosure. The user interface 600 is associated with a scenario creator tool. To ensure lasting relevance, the simulator may include a scenario builder shown in the user interface 600. The scenario builder enables a user or evaluator/instructor to create scenarios and share the scenario with others. A user may use the scenario creator tool for creating dialogue options for each turn in a scenario and assigning scores to each dialogue option. The user may also adjust attributes (e.g., attitude, goals, and mannerism) of the AI virtual companion. As depicted in FIG. 6, the user may input factors for a scenario related to lying in the “Scenario Input Selector” including guilt, statements, demeanor, and reaction of the AI virtual companion.

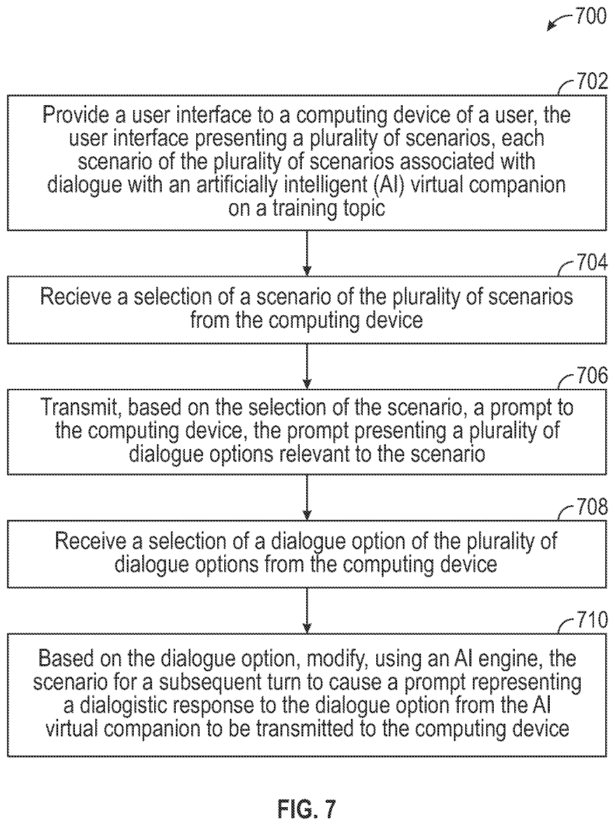

FIG. 7 illustrates example operations of a method 700 for using dialogue simulations for training according to certain embodiments of this disclosure. The method 700 may be performed by processing logic that may include hardware (circuitry, dedicated logic, etc.), software, or a combination of both. The method 700 and/or each of their individual functions, subroutines, or operations may be performed by one or more processors of a computing device (e.g., any component (server 128, etc.) of cloud-based computing system 116, or the computing device 102, of FIG. 1) implementing the method 700. The method 700 may be implemented as computer instructions stored on a memory device and executable by the one or more processors. In certain implementations, the method 700 may be performed by a single processing thread. Alternatively, the method 700 may be performed by two or more processing threads, each thread implementing one or more individual functions, routines, subroutines, or operations of the methods.

At block 702, the processing device may provide a user interface to a computing device of a user, where the user interface presents a plurality of scenarios and each scenario of the plurality of scenarios is associated with dialogue with an artificially intelligent (AI) virtual companion on a training topic. The computing device may include desktop computers, laptop computers, mobile devices (e.g., smartphones), tablet computers, etc. For example, the user interface may present a plurality of scenarios (e.g., lying, cheating, stealing, etc.) and each scenario of the plurality of scenarios is associated with dialogue with an artificially intelligent (AI) virtual companion on a training topic (e.g., honor, diversity and inclusion, and leadership).

At block 704, the processing device may receive a selection of a scenario of the plurality of scenarios from the computing device. The selection may include a selection of the scenario. In some embodiments, the user may also be provided a description of the scenario.

At block 706, the processing device may transmit, based on the selection of the scenario, a prompt to the computing device. The prompt may present a plurality of dialogue options (such as: confrontation, for example, “I saw you cheating”; inquiry, for example, “Tell me what happened”; and investigation, for example, “Did you know why you are here?”) relevant to the scenario (e.g., cheating).

At block 708, the processing device may receive a selection of a dialogue option of the plurality of dialogue options from the computing device.

At block 710, based on the dialogue option, the processing device may modify, using the AI engine 140, the scenario for a subsequent turn to cause a prompt representing a dialogistic response, to the dialogue option, from the AI virtual companion to be transmitted to the computing device. In some embodiments, the processing device may generate, via the AI engine 140, one or more machine learning models 132 trained to modify the scenario for the subsequent turn to cause a prompt to be transmitted to the computing device. In some embodiments, the AI engine 140 may include an expert system that includes rules and responses to the dialogue options. The expert system may use the rules and responses to modify the scenario for the subsequent turn to cause the prompt to be transmitted to the set of computing devices.

In some embodiments, the processing device may receive, from a sensor, one or more measurements pertaining to the user (e.g., heartrate), where the one or more measurements are received during the scenario, and the one or more measurements may indicate a characteristic of the user (e.g., an elevated heart rate may indicate that the user is stressed). The sensor may be a wearable device, such as a watch, a necklace, an anklet, a ring, a belt, etc. The sensor may include one or more devices for measuring any suitable characteristics of the user. Further, based on the characteristic, the processing device may modify, using the AI engine 140, the scenario for the subsequent turn (e.g., by avoiding combative dialogue). For example, the characteristics may comprise any of the following: a vital sign, a physiological state, a heartrate, a blood pressure, a pulse, a temperature, a perspiration rate, or some combination thereof. The sensor may include a wearable device, a camera, a device located proximate the user, a device included in the computing device, or some combination thereof.

In some embodiments, the simulator may include an interactive, virtual reality simulator configured to improve one-to-one communication by allowing users to practice conversations on difficult topics with a virtual, AI-powered companion while providing an evaluation of performance and analytics. For example, the simulator may empower USAFA cadets and instructors to practice conversations related to honor, and more specifically, how to confront a potential honor violation. The simulator may include an adaptive conversational engine (e.g., AI engine 140) and an AI virtual companion that responds to verbal input using speech-to-text language processing and natural language processing. The simulator may further include a virtual reality environment that allows users to view and interact with the AI virtual companion (e.g., by viewing, understanding, and responding to signs of agitation or distress) and a conversation library where content can be accessed, shared, and customized (e.g., users may adjust how the AI virtual companion responds to different inputs). Additionally, the simulator may include a post-conversation evaluation and analytics system that enables users to compare their approach with a community or against an optimal result (or receive a certification). In particular, the simulator is an interactive dialogue-driven trainer that may use a blend of virtual reality and natural language processing (including voice recognition via speech-to-text) to empower individuals to improve communication and collaboration. This is accomplished by users privately rehearsing simulated mission-essential conversations with an AI virtual companion (with customizable behaviors, goals, and mannerisms) on topics related to honor, diversity/equity/inclusion, and leadership. In accordance with embodiments disclosed herein, users may access the simulator using a commercial virtual reality device (e.g., Meta Quest®, Sony® PlayStation VR®, etc.,), and the user may select a conversation topic from a variety of learning objectives. The user may receive an overview of a learning objective (e.g., by reviewing relevant documentation such as a plagiarized paper, reading investigatory reports, or watching a witness video) and the desired outcomes. For example, the simulator may start, and the user may begin conversing with an AI-enabled virtual companion that understands what the user says into a microphone and responds with contextually accurate comments, answers, and questions. The AI virtual companion's response may be controlled by an expert AI system (e.g., AI engine 140 in FIG. 1) which balances several factors such as the user's attitude (e.g., friendly, angry, etc.,) and conversational choices, the virtual companion's characteristics, and background of the scenario to influence the AI virtual companion's behavior and the course of the conversation. Additionally, the user may see how the virtual companion reacts including body language and facial expressions through the virtual reality device. Further, once the scenario is complete, the user may review his or her choices, receive an evaluation, review analytics related to the conversation, and play the scenario again which allows the user to make different choices that can create different outcomes.

Other features the simulator may include are the following: diverse virtual companions; customizable “behaviors” of the virtual companion which provide users with exposure to a range of emotional responses (e.g., angry, quiet); the virtual companion having realistic facial expressions and non-verbal cues displayed in a virtual environment; conversations pulled directly from real-life (e.g., cadet honor violations) locations and interpersonal challenges; and replayable experiences by adjusting the virtual companion's behavior, choices, and background. Further, the technical improvements of the embodiments described herein include: (1) a user interface layer via virtual reality or a mobile device that receives spoken word, (2) speech to text conversion to enable understanding of the spoken word of a user, (3) natural language processing to assign meaning to the spoken word of a user, (4) an expert AI system to empower the AI virtual companion interactions, and (5) simulation and animation appropriate to the scenario and interactions between the user and the AI virtual companion.

Further, the simulator may serve as a flexible and customizable virtual reality conversational training tool. For example, within the context of training at the USAFA, the simulator enables honor training and is customizable with relevant scenarios (e.g., an honor code violation). The simulator may also empower instructors and students to improve difficult one-on-one communication, for example, by using realistic, simulated conversations focused on honor but extensible to leadership and diversity, equity, and inclusion (DEI). Additional advantages of the simulator include generating performance data on students and instructors related to “soft skills,” reinforcing the values of ethical leadership, and measuring quantitative improvement of users. Other advantages of the simulator include: producing an intuitive and accurate simulator user experience which may be customized (e.g., training scenarios for the USAFA); providing an easily learned interface and input/outputs that require little or no training to use; and producing an authentic environment and conversational companion including designs that are “true to life.”

In particular, within the context of training candidates at the USAFA, the simulator may produce a virtual reality environment that replicates an instructor's office, integrate educational/curriculum guidance as needed, and provide contextual and relevant learning as needed for the scenario (e.g., honor program considerations: “toleration” and “honor clarifications”). Additionally, the simulator may include adjustable AI virtual companion behavioral characteristics and each scenario may be associated with at least one designated conversational companion, one or more conditions (e.g., companion behavioral characteristics), and one or more outcomes.

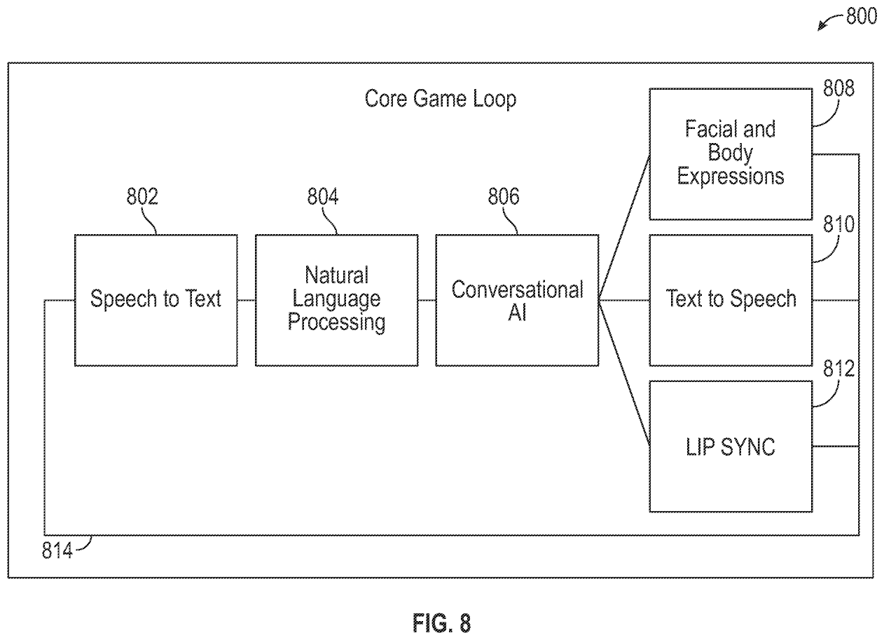

To explore the foregoing in more detail, FIG. 8 will now be described. FIG. 8 illustrates a high-level component diagram of an illustrative system architecture according to certain embodiments of this disclosure. FIG. 8 provides another exemplary embodiment of cloud-based computing system 116 in FIG. 1. As shown in FIG. 8, simulator 800 may include: a speech to text component 802 that is configured to record, analyze, and translate a user's voice input into text format; a natural language processing component 804 configured to analyze the user's input to generate a natural language understanding result; a AI virtual companion 806 configured to respond to the user's input; a facial and body expressions component 808 configured to determine reaction of AI virtual companion 806 to the user's voice input (e.g., based on a user's action and graphical representation of mood); a text to speech component 810 configured to transform responses of AI virtual companion into verbal replies; a lip synchronization component 812 configured to synchronize the visual representation of a mouth of AI virtual companion 806 to verbal responses; and core came loop 814 comprises multiple scenarios and branching narratives based on the response of AI virtual companion 806 to the user's input. Alternatively, or in addition to, simulator 800 may respond to a user's input in text or prerecorded responses.

In some embodiments, speech to text component 802 may receive speech audio data from a virtual reality device (e.g., computing device 102 in FIG. 1) and process the speech audio data and provides the text equivalent to natural language processing component 804. Speech to text component 802 may use one or more speech to text techniques to process the speech audio data. For example, models in speech recognition may be divided into an acoustic model and a language model. The acoustic model may solve the problem of turning sound signals into some kind of phonetic representation. The language model may house the domain knowledge of words, grammar, and sentence structure for the language. These conceptual models can be implemented with probabilistic models (e.g., Hidden Markov models, Deep Neural Network models, etc.,) using machine learning algorithms.

Further, natural language processing component 804 may use natural language processing (NLP), data mining, and pattern recognition technologies to process the text equivalent to generate a natural language understanding result. More specifically, natural language processing component 804 may use different AI technologies to understand language, translate content between languages, recognize elements in speech, and perform sentiment analysis. For example, natural language processing component 804 may use NLP and data mining and pattern recognition technologies to collect and process information provided in different information resources. Additionally, natural language processing component 804 may use natural language understanding (NLU) techniques to process unstructured data using text analytics to extract entities, relationships, keywords, semantic roles, and so forth. Natural language processing component 804 may generate the natural language understanding result to help AI engine 140 to understand the user's voice input. AI engine 140 may determine, based on the natural language understanding result, a response to the user's verbal input. In addition, using facial and body expressions component 808, test to speech component 810, and lip synchronization component 812, AI engine 140 may control visual content associated with the scenario being rendered on the display of the virtual reality device by rendering a representation of the AI virtual companion enacting a natural language response to the user's verbal input.

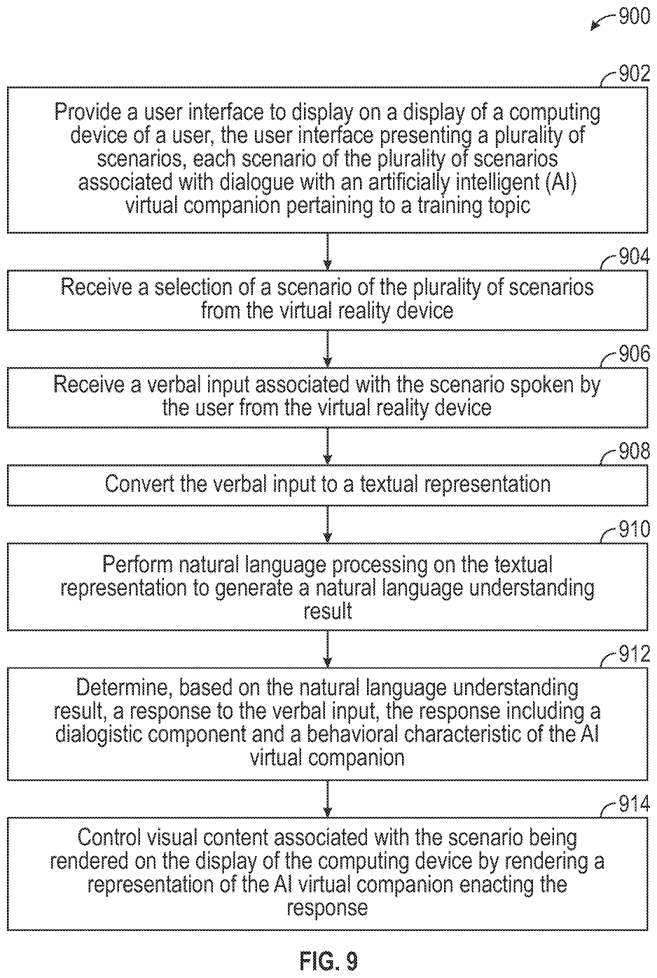

FIG. 9 illustrates example operations of a method 900 for using dialogue simulations for training according to certain embodiments of this disclosure. The method 900 may be performed by processing logic that may include hardware (circuitry, dedicated logic, etc.), software, or a combination of both. The method 900 and/or each of their individual functions, subroutines, or operations may be performed by one or more processors of a computing device (e.g., any component (server 128) of cloud-based computing system 116, or the computing device 102, of FIG. 1) implementing the method 900. The method 900 may be implemented as computer instructions stored on a memory device and executable by the one or more processors. In certain implementations, the method 900 may be performed by a single processing thread. Alternatively, the method 900 may be performed by two or more processing threads, each thread implementing one or more individual functions, routines, subroutines, or operations of the methods.

At block 902, the processing device may provide a user interface to a computing device of a user, where the user interface presents a plurality of scenarios and each scenario of the plurality of scenarios is associated with dialogue with an artificially intelligent (AI) virtual companion on a training topic. For example, the user interface may present a plurality of scenarios (e.g., lying, cheating, stealing, etc.) and each scenario of the plurality of scenarios is associated with dialogue with an artificially intelligent (AI) virtual companion on a training topic (e.g., honor, diversity and inclusion, and leadership).

At block 904, the processing device may receive a selection of a scenario of the plurality of scenarios from the computing device. The selection may include a selection of the scenario. In some embodiments, the user may also be provided a description of the scenario.

At block 906, the processing device may receive a verbal input associated with the scenario spoken by the user from the computing device. For example, a user's verbal input may include a user's confession or denial of an event relevant to the scenario, for example, on cheating.

At block 908, the processing device may convert the verbal input to textual representation. For example, speech to text component 802 in FIG. 8 may receive speech audio data from a computing device (e.g., computing device 102 in FIG. 1) and process the speech audio data and generate a text equivalent.

At block 910, the processing device may perform natural language processing on the textual representation to generate a natural language understanding result. For example, natural language processing component 804 may use NLP technologies to process the text equivalent to generate a natural language understanding result.

At block 912, the processing device may determine, based on the natural language understanding result, a response to the verbal input, where the response including a dialogistic component and a behavioral characteristic of the AI virtual companion. For example, AI engine 140 may determine, based on the natural language understanding result, a response to the user's verbal input. To help illustrate, in response to a scenario pertaining to cheating, AI engine 140 may determine to respond in an accusatory manner, for example, by telling the user: “I saw you cheating.” As another example, AI engine 140 may determine to respond in an investigatory fashion, for example, by asking the user: “Did you know why you are here?” The AI virtual companion may respond based on the user's attitude (e.g., accusatory, friendly, angry, etc.) and conversational choices. The AI virtual companion provides realistic conversational behavior with customizable behavioral characteristics (attitude, goals, and mannerism). For example, a behavioral characteristic may include a range of emotional responses (e.g., angry, sullen, quiet of the AI virtual companion. Additionally, the behavioral characteristics may include realistic facial expressions and non-verbal cues.

At block 914, the processing device may control visual content associated with the scenario being rendered on the display of the computing device by rendering a representation of the AI virtual companion enacting the response. For example, AI engine 140 may control visual content associated with the scenario being rendered on the display of the computing device by rendering a representation of the AI virtual companion enacting a natural language response to the user's verbal input.

Moreover, it is important that location and context of an interaction between individuals pertaining to a scenario is represented in the training provided by the simulator. For example, say in one scenario a USAFA cadet misses a meeting and an accountability cadet goes to determine why the USAFA cadet missed the meeting. If the cadet who missed the meeting says that he or she were sick and on bedrest but forgot to submit the form, where and how he or she says this impacts the response of the accountability cadet. If the conversation between the cadet and accountability cadet takes place in the cadet's room, the cadet is in a robe, and medicine is spotted on a nightstand, then the cadet is most likely sick and no further inquiry is need from the accountability cadet. However, if the conversation takes place on a sports field and the cadet is participating in a sport, then further inquiry is likely need from the accountability cadet.

In some embodiments, by the simulator implementing virtual reality, users are immersed in their surroundings. This allows users to better perceive and investigate their environments and incorporate these details into their analysis. In some embodiments, the simulator may implement augmented reality, and users' real environments may serve as a location of and context for an interaction between individuals pertaining to a scenario.

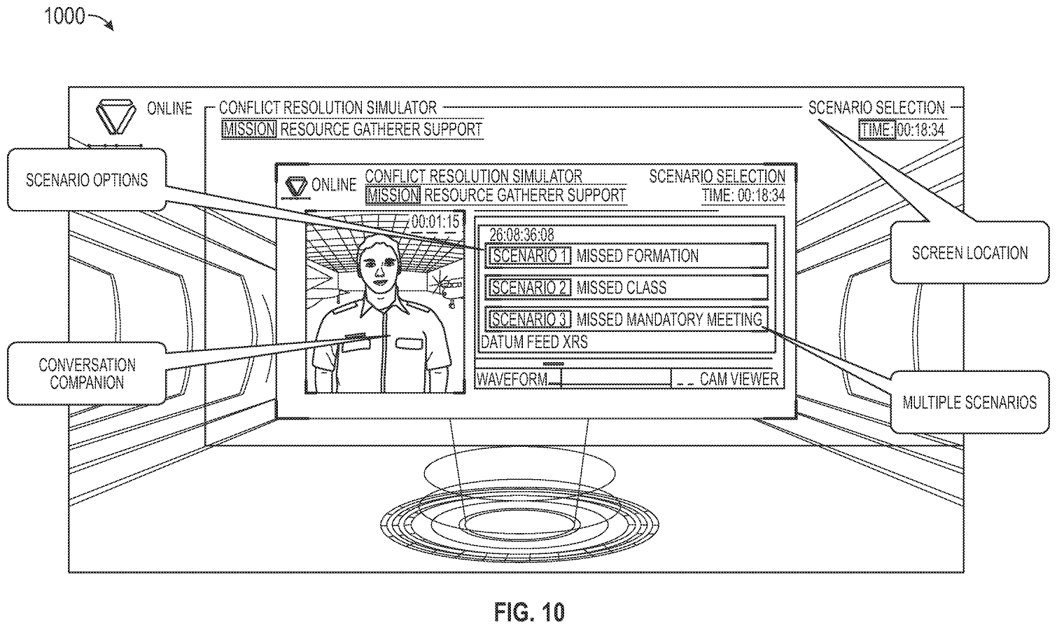

FIGS. 10-14 illustrate example virtual reality user interfaces of the simulator according to certain embodiments of this disclosure. FIG. 10 illustrates an example user interface 1000 of a virtual reality device for a starting screen of the conflict resolution simulator according to certain embodiments of this disclosure. The user interface 1000 presents a scenario selection screen that displays multiple scenarios (e.g., missed formation, missed class, missed mandatory meeting, etc.). The user may select from among multiple scenario options depending on their learning objective. The selection of the scenario may be transmitted to the cloud-based computing system 116. As shown in FIG. 10, the user interface 1000 displays a selectable AI virtual companion. In some embodiments, each scenario may be customizable by selecting different AI-enabled conversation companions, locations, and underlying fact patterns, which can influence the AI virtual companion's response.

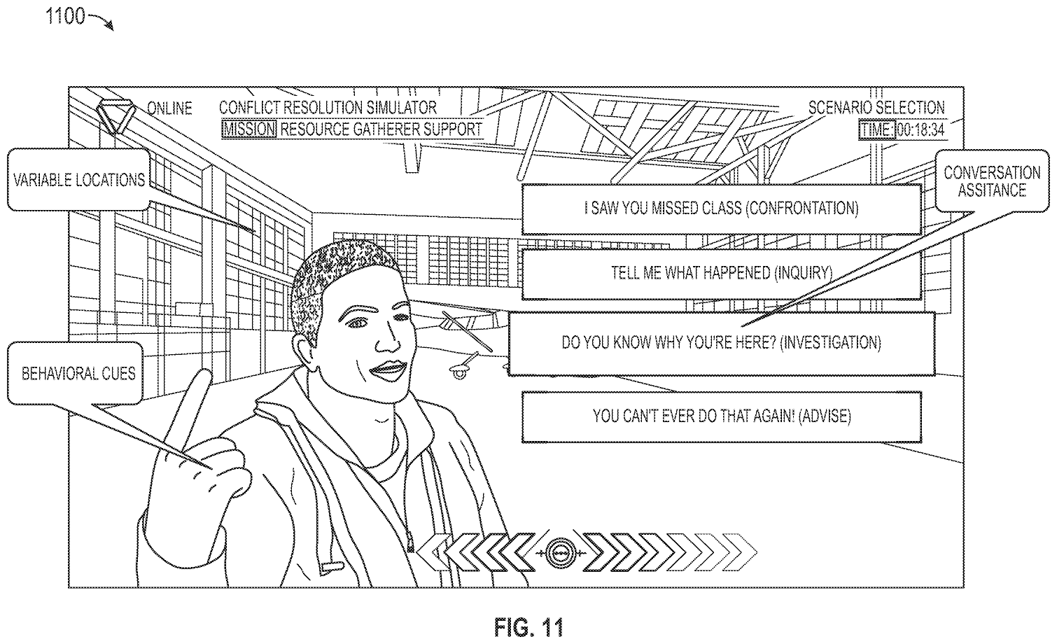

FIG. 11 illustrates an example user interface 1100 for a scenario execution screen according to certain embodiments of this disclosure. As depicted, the user interface 1100 presents an AI virtual companion. For example, after a cadet selects a scenario (e.g., missed class) in the user interface 1000 in FIG. 10, the simulator starts. As further depicted in FIG. 11, the user may begin interacting with one or more virtual companions. During the simulation, the AI virtual companion may respond to a user's verbal input. The virtual reality environment allows users to view and interact with the AI virtual companion (e.g., by viewing, understanding, and responding to signs of agitation or distress). Through the user interface 1100, the user may observe behavioral cues of the AI virtual companion and the virtual reality environment (e.g., location). The AI virtual companion may also display different behavioral cues (e.g., nerves, afraid, threatened, etc.), and the interaction may take place in different locations (e.g., a classroom, dorm room, sports field, etc.). In some embodiments, the AI virtual companion may respond based on the user's attitude (e.g., accusatory, friendly, angry, etc.) and conversational choices. Further, the AI virtual companion's programmed characteristics and scenario background influences the AI virtual companion's behavior and the course of the virtual conversation.

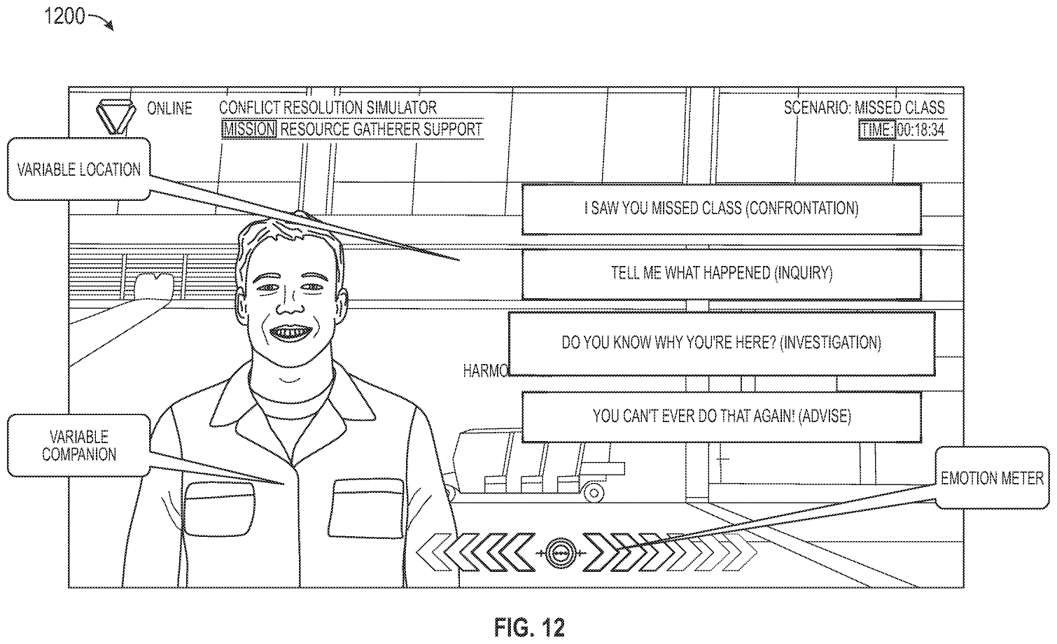

FIG. 12 illustrates another example user interface 1200 for a scenario execution screen according to certain embodiments of this disclosure. As shown in FIG. 12, in some embodiments, the AI virtual companion may include adjustable “behavioral” elements so that users face a range of emotional responses (e.g., angry, sullen, quiet). For example, in FIG. 12, the user may adjust the emotional state of the AI virtual companion through an emotion meter.

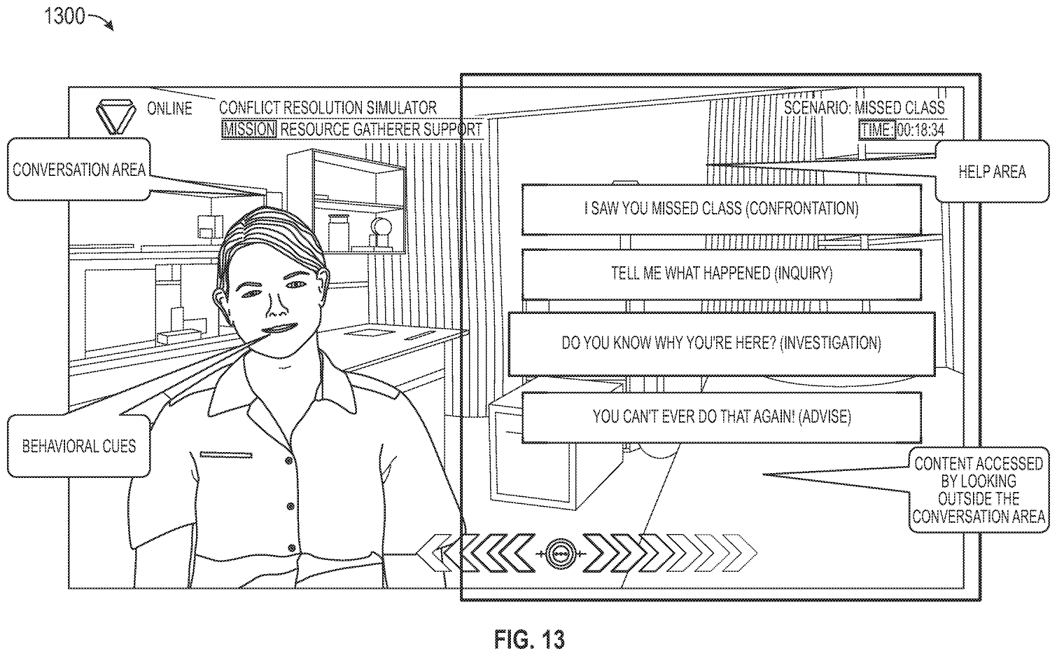

FIG. 13 illustrates another example user interface 1300 for a scenario execution screen according to certain embodiments of this disclosure. As shown in FIG. 13, conversation assistance may be provided to the user. The conversation assistance may provide conversation “suggestions” to the user (e.g., “Do you know why you're here?”). The user may select a conversation suggestion based on the training object (e.g., confrontation, inquiry, investigation, advise, etc.). In a virtual reality environment, the field of view may be adjusted by a user by turning his or her head. Accordingly, in some embodiments, the conversation suggestions may be viewable outside of the user's field of view when talking to the AI virtual companion but are available to the user by the user turning his or her head. For example, as shown in FIG. 13, the dialogue choice selected by the user may include “Do you know why you're here?”

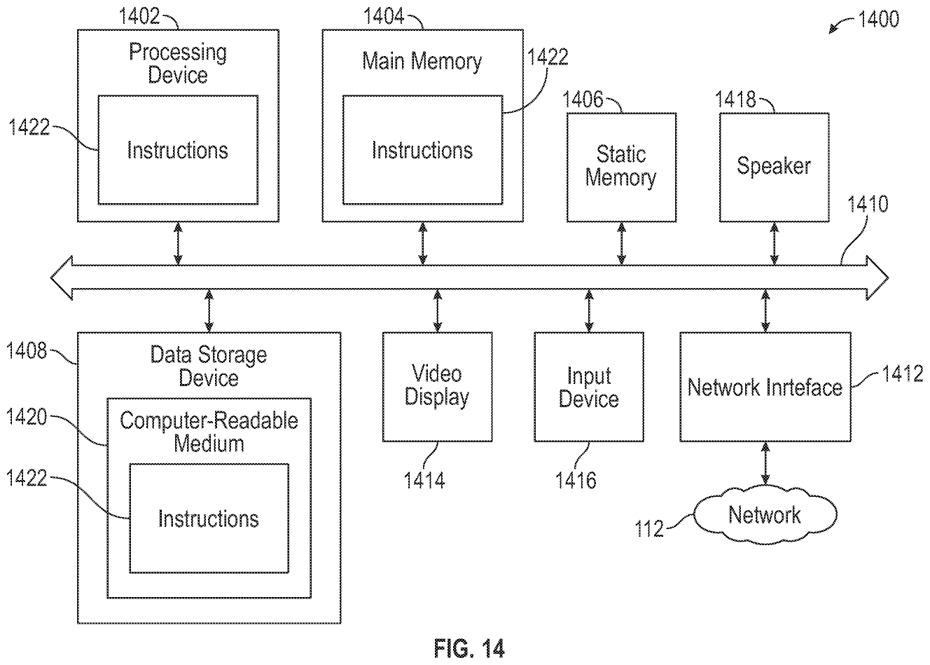

FIG. 14 illustrates an example computer system 1400, which can perform any one or more of the methods described herein. In one example, computer system 1400 may correspond to the computing device 102 or the one or more servers 128 of the cloud-based computing system 116 of FIG. 1. The computer system 1400 may be capable of executing the application 107 (e.g., scenario exercise platform) of FIG. 1. The computer system 1400 may be connected (e.g., networked) to other computer systems in a LAN, an intranet, an extranet, or the Internet. The computer system 1400 may operate in the capacity of a server in a client-server network environment. The computer system 1400 may be a personal computer (PC), a tablet computer, a laptop, a wearable (e.g., wristband), a set-top box (STB), a personal Digital Assistant (PDA), a smartphone, a camera, a video camera, or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The computer system 1400 includes a processing device 1402, a main memory 1404 (e.g., read-only memory (ROM), solid state drive (SSD), flash memory, dynamic random access memory (DRAM) such as synchronous DRAM (SDRAM)), a static memory 1406 (e.g., solid state drive (SSD), flash memory, static random access memory (SRAM)), and a data storage device 1408, which communicate with each other via a bus 1410.

Processing device 1402 represents one or more general-purpose processing devices such as a microprocessor, central processing unit, or the like. More particularly, the processing device 1402 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 1402 may also be one or more special-purpose processing devices such as an application specific integrated circuit (ASIC), a field programmable gate array (FPGA), a digital signal processor (DSP), network processor, or the like. The processing device 1402 is configured to execute instructions for performing any of the operations and steps discussed herein.

The computer system 1400 may further include a network interface device 1412. The computer system 1400 also may include a video display 1414 (e.g., a liquid crystal display (LCD) or a cathode ray tube (CRT)), one or more input devices 1416 (e.g., a keyboard and/or a mouse), and one or more speakers 1418 (e.g., a speaker). In one illustrative example, the video display 1414 and the input device(s) 1416 may be combined into a single component or device (e.g., an LCD touch screen).

The data storage device 1416 may include a computer-readable medium 1420 on which the instructions 1422 (e.g., implementing the application 107, and/or any component depicted in the FIGURES and described herein) embodying any one or more of the methodologies or functions described herein are stored. The instructions 1422 may also reside, completely or at least partially, within the main memory 1404 and/or within the processing device 1402 during execution thereof by the computer system 1400. As such, the main memory 1404 and the processing device 1402 also constitute computer-readable media. The instructions 1422 may further be transmitted or received over a network via the network interface device 1412.

While the computer-readable storage medium 1420 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

A method for using dialogue simulations for training, the method comprises: providing a user interface to display on a display of a computing device of a user, the user interface presenting a plurality of scenarios, each scenario of the plurality of scenarios associated with dialogue with an artificially intelligent (AI) virtual companion pertaining to a training topic; receiving a selection of a scenario of the plurality of scenarios from the computing device; receiving a verbal input associated with the scenario spoken by the user from the computing device; converting the verbal input to a textual representation; performing natural language processing on the textual representation to generate a natural language understanding result; determining, based on the natural language understanding result, a response to the verbal input, the response including a dialogistic component and a behavioral characteristic of the AI virtual companion; and controlling visual content associated with the scenario being rendered on the display of the computing device by rendering a representation of the AI virtual companion enacting the response.

The foregoing method further comprises determining the response to the verbal input based on at least one of the following: an attitude of the user, conversational choices of the user, the behavioral characteristic of the AI virtual companion, and background information of the scenario.

The foregoing method where the training topic is related to one of the following topics: honor, diversity and inclusion, and leadership.

The foregoing method further comprises providing background information on the training topic.

The foregoing method further comprises providing a user interface configured to allow adjustment of the behavioral characteristic of the AI virtual companion.

The foregoing method further comprises providing a user interface configured to allow adjustment of the dialogistic component of the AI virtual companion.

The foregoing method further comprises providing a user interface configured to allow the user to playback the scenario and review one or more selections made by the user during the scenario.

The foregoing method further comprises providing a user interface configured to allow the user to review one or more selections of other users for the scenario.

The foregoing method further comprises providing a user interface configured to allow the user to create dialogue and one or more outcomes for a new scenario.

The foregoing method further comprises receiving, from a sensor, one or more measurements pertaining to the user, wherein the one or more measurements are received during the scenario, and the one or more measurements indicate a characteristic of the user; and based on the characteristic, modifying the visual content associated with the scenario being rendered on the display of the computing device.

The foregoing method where the sensor is a wearable device, a camera, a device located proximate the user, a device included in the computing device, or some combination thereof.

The foregoing method where the characteristic comprises a vital sign, a physiological state, a heartrate, a blood pressure, a pulse, a temperature, a perspiration rate, or some combination thereof.

A tangible, non-transitory computer-readable medium storing instructions that, when executed, cause a processing device to: provide a user interface to display on a display of a computing device of a user, the user interface presenting a plurality of scenarios, each scenario of the plurality of scenarios associated with dialogue with an artificially intelligent (AI) virtual companion pertaining to a training topic; receive a selection of a scenario of the plurality of scenarios from the computing device; receive a verbal input associated with the scenario spoken by the user from the computing device; convert the verbal input to a textual representation; perform natural language processing on the textual representation to generate a natural language understanding result; determine, based on the natural language understanding result, a response to the verbal input, the response including a dialogistic component and a behavioral characteristic of the AI virtual companion; and control visual content associated with the scenario being rendered on the display of the computing device by rendering a representation of the AI virtual companion enacting the response.

The foregoing computer-readable medium wherein the processing device is further caused to determine the response to the verbal input based on at least one of the following: an attitude of the user, conversational choices of the user, the behavioral characteristic of the AI virtual companion, and background information of the scenario.

The foregoing computer-readable medium, wherein the training topic is related to one of the following topics: honor, diversity and inclusion, and leadership.

The foregoing computer-readable medium, wherein the processing device is further caused to provide background information on the training topic.

The foregoing computer-readable medium, wherein the processing device is further caused to provide a user interface configured to allow adjustment of the behavioral characteristic of the AI virtual companion.

The foregoing computer-readable medium, wherein the processing device is further caused to provide a user interface configured to allow adjustment of the dialogistic component of the AI virtual companion.

The foregoing computer-readable medium, wherein the processing device is further caused to provide a user interface configured to allow the user to playback the scenario and review one or more selections made by the user during the scenario.

A system comprising: a memory device storing instructions; a processing device communicatively coupled to the memory device, wherein the processing device executes the instructions to: provide a user interface to display on a display of a computing device of a user, the user interface presenting a plurality of scenarios, each scenario of the plurality of scenarios associated with dialogue with an artificially intelligent (AI) virtual companion pertaining to a training topic; receive a selection of a scenario of the plurality of scenarios from the computing device; receive a verbal input associated with the scenario spoken by the user from the computing device; converting the verbal input to a textual representation; perform natural language processing on the textual representation to generate a natural language understanding result; determine, based on the natural language understanding result, a response to the verbal input, the response including a dialogistic component and a behavioral characteristic of the AI virtual companion; and control visual content associated with the scenario being rendered on the display of the computing device by rendering a representation of the AI virtual companion enacting the response.

The various aspects, embodiments, implementations or features of the described embodiments can be used separately or in any combination. The embodiments disclosed herein are modular in nature and can be used in conjunction with or coupled to other embodiments, including both statically-based and dynamically-based equipment. In addition, the embodiments disclosed herein can employ selected equipment such that they can identify individual users and auto-calibrate threshold multiple-of-body-weight targets, as well as other individualized parameters, for individual users.

1. A method for using dialogue simulations for training, the method comprising:
providing a user interface to display on a display of a computing device of a user, the user interface presenting a plurality of scenarios, each scenario of the plurality of scenarios associated with dialogue with an artificially intelligent (AI) virtual companion pertaining to a training topic;
receiving a selection of a scenario of the plurality of scenarios from the computing device;
receiving a verbal input associated with the scenario spoken by the user from the computing device;
converting the verbal input to a textual representation;
performing natural language processing on the textual representation to generate a natural language understanding result;
determining, based on the natural language understanding result, a response to the verbal input, the response including a dialogistic component and a behavioral characteristic of the AI virtual companion; and
controlling visual content associated with the scenario being rendered on the display of the computing device by rendering a representation of the AI virtual companion enacting the response.

2. The method of claim 1, further comprising determining the response to the verbal input based on at least one of the following: an attitude of the user, conversational choices of the user, the behavioral characteristic of the AI virtual companion, and background information of the scenario.

3. The method of claim 1, wherein the training topic is related to one of the following topics: honor, diversity and inclusion, and leadership.

4. The method of claim 1, further comprising providing background information on the training topic.

5. The method of claim 1, further comprising providing a user interface configured to allow adjustment of the behavioral characteristic of the AI virtual companion.

6. The method of claim 1, further comprising providing a user interface configured to allow adjustment of the dialogistic component of the AI virtual companion.

7. The method of claim 1, further comprising providing a user interface configured to allow the user to playback the scenario and review one or more selections made by the user during the scenario.

8. The method of claim 1, further comprising providing a user interface configured to allow the user to review one or more selections of other users for the scenario.

9. The method of claim 1, further comprising providing a user interface configured to allow the user to create dialogue and one or more outcomes for a new scenario.

10. The method of claim 1, further comprising:
receiving, from a sensor, one or more measurements pertaining to the user, wherein the one or more measurements are received during the scenario, and the one or more measurements indicate a characteristic of the user; and
based on the characteristic, modifying the visual content associated with the scenario being rendered on the display of the computing device.

11. The method of claim 10, wherein the sensor is a wearable device, a camera, a device located proximate the user, a device included in the computing device, or some combination thereof.

12. The method of claim 10, wherein the characteristic comprises a vital sign, a physiological state, a heartrate, a blood pressure, a pulse, a temperature, a perspiration rate, or some combination thereof

13. A tangible, non-transitory computer-readable medium storing instructions that, when executed, cause a processing device to:
provide a user interface to display on a display of a computing device of a user, the user interface presenting a plurality of scenarios, each scenario of the plurality of scenarios associated with dialogue with an artificially intelligent (AI) virtual companion pertaining to a training topic;
receive a selection of a scenario of the plurality of scenarios from the computing device;
receive a verbal input associated with the scenario spoken by the user from the computing device;
convert the verbal input to a textual representation;
perform natural language processing on the textual representation to generate a natural language understanding result;
determine, based on the natural language understanding result, a response to the verbal input, the response including a dialogistic component and a behavioral characteristic of the AI virtual companion; and
control visual content associated with the scenario being rendered on the display of the computing device by rendering a representation of the AI virtual companion enacting the response.

14. The computer-readable medium of claim 13, wherein the processing device is further caused to determine the response to the verbal input based on at least one of the following: an attitude of the user, conversational choices of the user, the behavioral characteristic of the AI virtual companion, and background information of the scenario.

15. The computer-readable medium of claim 13, wherein the training topic is related to one of the following topics: honor, diversity and inclusion, and leadership.

16. The computer-readable medium of claim 13, wherein the processing device is further caused to provide background information on the training topic.

17. The computer-readable medium of claim 13, wherein the processing device is further caused to provide a user interface configured to allow adjustment of the behavioral characteristic of the AI virtual companion.

18. The computer-readable medium of claim 13, wherein the processing device is further caused to provide a user interface configured to allow adjustment of the dialogistic component of the AI virtual companion.

19. The computer-readable medium of claim 13, wherein the processing device is further caused to provide a user interface configured to allow the user to playback the scenario and review one or more selections made by the user during the scenario.

20. A system comprising:
a memory device storing instructions;
a processing device communicatively coupled to the memory device, wherein the processing device executes the instructions to:
provide a user interface to display on a display of a computing device of a user, the user interface presenting a plurality of scenarios, each scenario of the plurality of scenarios associated with dialogue with an artificially intelligent (AI) virtual companion pertaining to a training topic;
receive a selection of a scenario of the plurality of scenarios from the computing device;
receive a verbal input associated with the scenario spoken by the user from the computing device;
convert the verbal input to a textual representation;
perform natural language processing on the textual representation to generate a natural language understanding result;
determine, based on the natural language understanding result, a response to the verbal input, the response including a dialogistic component and a behavioral characteristic of the AI virtual companion; and
control visual content associated with the scenario being rendered on the display of the computing device by rendering a representation of the AI virtual companion enacting the response.
